(19) United States
(12) Patent Application Publication
(10) Pub. No.: US 2007/0110666 A1
(43) Pub. Date: May 17, 2007

(54) Methods for preparation of live body tissues for examination

(76) Inventors: Paul Pevsner, New York, NY (US); Frederick Naftolin, Woodbridge, CT (US); Ahmed Fadiel Metwaly, New Haven, CT (US); Erol Gulcicek, Madison, CT (US)

Correspondence Address: KLAUBER & JACKSON, 411 HACKENSACK AVENUE, HACKENSACK, NJ, 07601, US

(21) Appl. No.: 11/541,403

(22) Filed: Sep. 28, 2006

Related U.S. Application Data
(60) Provisional application No. 60/722,207, filed on Sep. 30, 2005.
(60) Provisional application No. 60/724,585, filed on Oct. 7, 2005.

(51) Int. Cl. A61K 51/00; A01N 1/02; A61K 49/04; C12Q 1/00

(52) U.S. Cl. 424/1.11; 424/9.4; 435/1.1; 435/4

(57) ABSTRACT

A minimally invasive procedure for perfusion-impregnation of tissues, organs and cells is provided that aids in the retention of tissue and cellular architecture without the formation of artifacts. The procedure allows for monitoring drug disposition, for detecting diseased tissue and for monitoring the presence of target molecules in a sample using various imaging techniques, including matrix assisted laser desorption ionization mass spectrometry (MALDI-MS).

### CROSS-REFERENCE TO RELATED APPLICATIONS

This application is a non-provisional application, which claims priority to provisional application Ser. No. 60/722,207, filed Sep. 30, 2005 and to provisional application Ser. No. 60/724,585, filed Oct. 7, 2005, both of which are incorporated herein by reference in their entireties. Applicants claim the benefits of these applications under 35 U.S.C. §119(e).

### FIELD OF THE INVENTION

The present invention is directed to methods for impregnating live tissues, organs or cells of animals with substances that enable subsequent studies, including but not limited to pathological analyses or biochemical analyses. The methods incorporate the use of various fixatives, matrices, buffers, and other perfusates for optimizing the visualization of tissue, organ or cellular architecture without inducing artifacts that are often observed using other standard methodologies.

### BACKGROUND

Ischemia causes rapid destruction of tissues or cells after they have been deprived of oxygen for a certain length of time. More particularly, neurons and neuronal support structures in brain tissue are destroyed after an ischemic event that deprives neurons and neuronal tissue of oxygen. Anoxia immediately precipitates a cascade of events resulting in neuronal and neuropil necrosis (Srinivasan, M., D. Sedmak and S. Jewell. (2002), Effect of fixatives and tissue processing on the content and integrity of nucleic acids. Am J Pathol 161:1961-1971). Minimally disruptive fixation is necessary to preserve tissue ultrastructure and morphology for examination by light and electron microscopy. Structural integrity decreases over time as RNA and proteins degrade in tissue samples. Preservation of RNA and protein requires immediate snap-freezing, perfusion or immersion with normal saline (Vincek, V., M. Nassiri, J. Knowles, M. Nadji and A. R. Morales (2003), Preservation of tissue RNA in normal saline. Lab Invest 83:137-138). The negative effects of fixatives and fixation on immunohistochemical detection of RNA and antigenic proteins have been extensively reviewed in the literature (O'Leary, T. J. (2001), Standardization in Immunohistochemistry. Appl Immunohistochem Mol Morphol 9:3-8; Shi, S. R., R. J. Cote and C. R. Taylor. (2001), Antigen retrieval techniques: current perspectives. J Histochem Cytochem 49:931-937). A complete analysis of the ischemic cascade requires preservation of ultrastructure as well as RNA and protein.

Currently, the recommended techniques for brain fixation require thoracotomy of the animals and direct cardiac perfusion with fixative solution under pressure (Mammen, P. P. A., J. M. Shelton, S. C. Goetsch, S. C. Williams, J. A. Richardson, M. G. Garry and D. J. Garry. (2002), Neuroglobin, A Novel Member of the Globin Family, Is Expressed in Focal Regions of the Brain. J. Histochem. Cytochem. 50:1591-1598; Shelton, J. M., M. H. Lee, J. A. Richardson and S. B. Patel. (2000), Microsomal triglyceride transfer protein expression during mouse development. J Lipid Res 41:532-537; Hockfield, S., Carson S., et al. (1993), Selected Methods for Antibody and Nucleic Acid Probes, p. 6. In S. Hockfield (Ed.), Molecular Probes of the Nervous System. Cold Spring Harbor Laboratory Press, Plainview, N.Y.; Isope, P. and B. Barbour. (2002), Properties of Unitary Granule Cellright-arrowPurkinje Cell Synapses in Adult Rat Cerebellar Slices. J. Neurosci. 22:9668-9678; Krinke, G. J. (2000). The Laboratory Rat. Academic Press, Switzerland.

Fixation under pressure results in swelling of the extravascular space and dilation of the ventricles (Cragg, B. (1980) Preservation of extracellular space during fixation of the brain for electron microscopy. Tissue Cell 12:63-72). In this standard technique, the animal is placed under terminal anesthesia and is subjected to thoracotomy followed by cardiac perfusion with buffered saline. Sudden interruption of blood flow results in immediate anoxia and initiates the ischemic cascade (Srinivasan, M., D. Sedmak and S. Jewell. (2002), Effect of fixatives and tissue processing on the content and integrity of nucleic acids. Am J Pathol 161:1961-1971). Subsequently, the animal is perfused through the heart with fixative. The procedure requires approximately thirty minutes per animal. The procedure duration limits the number of experiments that can be performed.

The commonly employed technique of thoracotomy with left ventricular perfusion requires surgical instruments, tubing, connectors, switches, reservoirs, large quantities of reagents and several feet of bench space, and time (Mammen, P. P. A., J. M. Shelton, S. C. Goetsch, S. C. Williams, J. A. Richardson, M. G. Garry and D. J. Garry. (2002), Neuroglobin, A Novel Member of the Globin Family, Is Expressed in Focal Regions of the Brain. J. Histochem. Cytochem. 50:1591-1598; Shelton, J. M., M. H. Lee, J. A. Richardson and S. B. Patel. (2000), Microsomal triglyceride transfer protein expression during mouse development. J Lipid Res 41:532-537; Isope, P. and B. Barbour. (2002), Properties of Unitary Granule Cellright-arrowPurkinje Cell Synapses in Adult Rat Cerebellar Slices. J. Neurosci. 22:9668-9678).

Accordingly, there is a need for a minimally invasive procedure for live tissue perfusion that allows for maintaining the tissue, organ or cellular architecture without producing artifacts. The present disclosure provides for such methods.

The citation of any reference herein should not be construed as an admission that such reference is available as “Prior Art” to the instant application.

### SUMMARY OF THE INVENTION

In its broadest aspect, the present invention relates to a minimally-invasive technique for in vivo tissue, organ or cell perfusion in animals that uses the beating heart to circulate the fixative or other agents and therefore approaches physiological conditions during perfusion and/or fixation. The fixed tissue can be harvested in as little as 90 to 120 seconds. This technique allows for the preservation of cytomorphology and cellular ultrastructure and minimizes the formation of artifacts in the sample. This procedure thus allows for a more accurate diagnostic assessment of diseased tissues, organs or cellular abnormalities.

Accordingly, the invention provides for a minimally invasive method for preparation of live body tissues for examination, comprising transthoracic cardiac infusion of a tissue perfusate in an amount sufficient to preserve the ultrastructure of the tissue without inducing artifacts. In one particular embodiment, the technique is used for infusion of a material into a mammal for preservation of ultrastructure in brain tissue. In another particular embodiment, the mammal is a human. In another particular embodiment, the mammal is a non-human mammal, selected from a rodent, including rats, mice, hamsters and gerbils. In yet another particular embodiment, the mammal is a non-human primate, such as a monkey. In yet another particular embodiment, the non-human mammal is selected from rabbits, goats, sheep, swine, dogs, cats, and horses.

In a preferred embodiment, the minimally invasive method for live tissue perfusion comprises the steps of:
a) inserting a needle into the left ventricle of the heart through a percutaneous puncture of the left lateral chest wall at the juncture of the anterior at about ¼ to ½ of the thorax and the posterior at about ½ to ¾ of the thorax in the anterior/posterior plane and at the juncture of the superior ⅔ to ¾ and the inferior ⅓ to ½ of the distance between the axilla and the inferior margin of the rib cage in the cranio-caudal plane;
b) delivering a perfusate using the method of step (a) into an animal for a time period ranging from about 10 seconds to less than or equal to one minute;
c) removing a bodily tissue for analysis.

In a more preferred embodiment, the point of needle insertion is at the juncture of the anterior one third and posterior two thirds of the thorax in the anterior/posterior plane, and at the juncture of the superior two thirds and the inferior one third of the distance between the axilla and the inferior margin of the rib cage in the cranio-caudal plane. The preferred embodiments provide for left ventricular perfusion without the need for thoracotomy.

In another particular embodiment, the tissue perfusate is a fixative, a solvent, a matrix liquid for use in matrix assisted laser desorption ionization imaging (MALDI), a radionuclide, an imaging or contrast agent, a radiographic contrast medium, a cryopreservative, a biomarker, or a buffered solution for delivery of a therapeutic or diagnostic agent.

In another particular embodiment, the fixative is selected from the group consisting of an aldehyde, such as, but not limited to, paraformaldehyde, glutaraldehyde, formaldehyde and glyoxal. In yet another particular embodiment, the fixative is selected from the group consisting of an alcohol, including, but not limited to, ethanol, methanol, isopropanol, propanol, butanol, isobutanol, ethyl butane and amyl alcohol. In another particular embodiment, the fixative is selected from the group consisting of a ketone, including, but not limited to acetone or methyl ethyl ketone.

In yet another particular embodiment the buffered solution is selected from the group consisting of phosphate buffered saline (PBS), a phosphate buffer, a potassium buffer, a choline buffer and a glycine buffer.

In yet another particular embodiment the cryopreservative is selected from the group consisting of propylene glycol, ethylene glycol, trialose, sucrose, glycerol and a bisaccharide.

In yet another particular embodiment the matrix liquid or solvent for use in matrix assisted laser desorption ionization imaging (MALDI) is infused prior to or concurrent with the infusion of the fixative.

In yet another particular embodiment the matrix liquid is selected from the group consisting of α-4-cyano hydroxy cinnamic acid (CHCA), sinnapinic acid, a heavy metal and glycerol.

In yet another particular embodiment the method provides for perfusion of the perfusate at physiologic blood pressure and heart rate.

In yet another particular embodiment the method provides for uniform distribution and impregnation of perfusate throughout all tissues, organs and cells of the body, without distortion.

In yet another particular embodiment the method provides for targeting of a diagnostic or therapeutic agent to a tissue, cell or organ.

In yet another particular embodiment the MALDI is used for the imaging of a tissue, an organ, a cellular protein, a peptide, a sugar, a salt, an organic acid or any other low molecular weight molecule.

In yet another particular embodiment the solvent is dimethyl sulfoxide (DMSO), methanol, dimethyl formamide (DMF), polyethylene glycol, beta-mercaptoethanol, ethanol, propylene glycol, or ethylene glycol.

In yet another particular embodiment the method of further comprises infusing of a drug, a protein, an antibody, a nucleic acid, a carbohydrate or a lipid.

In yet another particular embodiment the drug, the protein, the antibody, the nucleic acid, the carbohydrate or the lipid is labeled for monitoring tissue, organ or cellular disposition or damage.

In yet another particular embodiment the drug, the protein, the antibody, the nucleic acid, the carbohydrate or the lipid is labeled with a marker selected from the group consisting of a radioisotope, a fluorophore, an enzyme or a heavy metal.

In yet another particular embodiment the examination of tissues may be performed by a method selected from the group consisting of light microscopy, electron microscopy, atomic microscopy, Magnetic Resonance Imaging (MRI), ultrasound, x-ray computed tomography (CT), single photon emission computed tomography (SPECT) and/or positron emission tomography (PET), mass spectrometry, matrix assisted laser desorption ionizing imaging (MALDI-MS) spectrometry, surface-enhanced laser desorption/ionization mass spectrometry (SELDI), in vivo biophotonic imaging (VivoVision) and any other imaging method suitable for studying organ, tissue or cellular ultrastructure.

In yet another particular embodiment the labeled drug, protein, antibody, nucleic acid, carbohydrate or lipid is uniformly distributed throughout the tissues, organs or cells of the body, before they are harvested for study.

Other aspects and advantages will become apparent from a review of the ensuing detailed description taken in conjunction with the following illustrative drawings.

### BRIEF DESCRIPTION OF THE DRAWINGS

FIG. 1. Illustration of Protocol for Infusion of the Perfusate

FIG. 2: Mouse brain histology stained with H&E and GFAP. A: H&E picric acid-paraformaldehyde-glutaraldehyde fixative (1.5×). B: H&E magnified from FIG. 1A shows preservation of neuropil and neuronal detail (100×). C: GFAP antibody shows preservation of glial structure (100×).

FIG. 3: A & B: Sequential unstained murine brain sections (50 micron). Note the two punch sampling sites for electron microscopy on section B.

FIG. 4: Electron microscopic (EM) images. Magnification levels are noted beneath each frame. Intact cytology and sub-cellular structures A: Neuron with euchromatic nucleus, cytoplasm rich with healthy mitochondria, apical dendrites, and neuropil with dendrites and axons. B: Neuronal nucleus and nucleolus. Rim of cytoplasm with mitochondria, dendrites and a few myelinated axons. C: Neuron with nucleus and cytoplasm containing mitochondria, rough endoplasmic reticulum and golgi apparatus. Peri-neuronal neuropil with dendrites and two myelinated axons.

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms a “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference in their entireties.

Definitions

The terms used herein have the meanings recognized and known to those of skill in the art, however, for convenience and completeness, particular terms and their meanings are set forth below.

The term “minimally invasive” refers to procedures, such as very small incisions or injections, which are used in order to minimize the damaging effects of large muscle retraction during surgical procedures. Minimally invasive procedures attempt to leave the body as naturally intact as it was prior to surgery. The goal is to achieve rapid recovery, lessen post-operative pain, and leave cosmetically satisfying incisional scars. In the context of the present invention, the term also takes into account the fact that a thoracotomy is not necessary to inject the materials into the left ventricle of the heart in order to achieve the desired effect.

The term “perfusate” or “tissue perfusate” refers to a liquid that has been passed over or through the vessels of an organ or tissue or cells.

An “amount sufficient to preserve the ultrastructure of a tissue without inducing artifacts” refers to the amount of perfusate introduced using the methods of the present invention that is sufficient to preserve the normal architecture of the cell, tissue or organ, while at the same time minimizing the introduction of artifacts into the preparation. For example, in the present invention, about 0.1 ml to about 1.0 ml of a tissue perfusate is the preferred range of material for injection, and more particularly, this relates to about 0.1 ml or more for fixatives such as 4% methyl or 4% ethyl alcohol and about 1.0 ml for cryprotectants, such as 5% sucrose. These values represent the amounts injected in a 25 g mouse and are modified accordingly as a percentage of body weight in larger species. In a particular embodiment, the term “about” means within 20%, preferably within 10%, and more preferably within 5%.

A “fixative”, as used herein, refers to a substance that is used to protect or preserve specimens of tissues, organs or cells.

The term “infusion” refers to the injection of fluid into a blood vessel in order to reach an organ or tissue.

The terms “contrast medium” and “contrast agent” are used interchangeably and refer to a substance, such as barium or air, used in radiography to increase the contrast of an image. A positive contrast medium absorbs x-rays more strongly than the tissue or structure being examined; a negative contrast medium, less strongly. The terms “contrast medium” or “contrast agent,” thus refers to an agent used to highlight specific areas so that organs, blood vessels, and/or tissues are more visible. By increasing the visibility of the surfaces being studied, the presence and extent of disease and/or injury can be determined

The term “biomarker” refers to a highly specific molecule, the existence and levels of which are causally connected to a complex biological process, and reliably captures the state of said process. Furthermore, a biomarker, to be of practical importance, should be present in samples that can be obtained from individuals without endangering their physical integrity or well-being.

The term “organic acid” refers to any of various acids containing one or more carbon-containing radicals.

The term “solvent” refers to a substance capable of dissolving another substance.

The term “low molecular weight molecule” refers to a molecule that is generally less than 2 Kd in molecular weight.

The term “transthoracic cardiac infusion” refers to the infusion of heart tissue by injecting a material across or through the thoracic cavity or chest wall.

The term “cryopreservative” refers to any material used to retain the stability of a sample, for example, a tissue, organ or cellular sample, when frozen.

The terms “MALDI” and “MALDI-MS” are used interchangeably and refer to matrix assisted laser desorption imaging and matrix assisted laser desorption/ionization mass spectrometry, which entails methods of mass spectrometric analysis which use a laser as a means to desorb, volatize, and ionize an analyte. In MALDI-MS methods, the analyte is contacted with a matrix material to prepare the analyte for analysis. The matrix material absorbs energy from the laser and transfers the energy to the analyte to desorb, volatize, and ionize the analyte, thereby producing ions from the analyte that are then analyzed in the mass spectrometer to yield information about the analyte.

A “matrix” or a “matrix liquid” refers to a material used in MALDI-MS to prepare the sample analyte for analysis. As noted above, this material absorbs energy from the laser and transfers the energy to the analyte to desorb, volatize, and ionize the analyte, thereby producing ions from the analyte that are then analyzed in the mass spectrometer to yield information about the analyte. Samples of such matrix materials or matrix liquids include, but are not limited to sinapinic acid (SA) and derivatives thereof, such as alpha-cyano sinapinic acid; cinnamic acid and derivatives thereof, such as α-4-cyano hydroxyl cinnamic acid (CHCA); 3,5-dimethoxy-4-hydroxycinnamic acid; 2,5-dihydroxybenzoic acid (DHB); and dithranol. Other examples include heavy metals and glycereol.

General Description

The present invention provides a minimally invasive method for impregnating tissues, organs or cells with a perfusate in preparation for further analysis, including, but not limited to, pathological examination by microscopy including, but not limited to light, electron and atomic microscopy. This injection method also provides for delivery of a perfusate to tissues, organs or cells for other types of analysis, such as, but not limited to, biochemical analysis, including but not limited to mass spectrometry. The method described herein is a method for percutaneous transcardiac injection of liquid in human and other living subjects, without the need for performing a thoracotomy. For purposes of example, the invention is described as it is performed using injection of liquid material into a living animal for the purpose of uniform, physiologic perfusion of that liquid material throughout the tissues, organs, and cells of the body. However, the impregnation is not dependent only on perfusion; nor, is it only limited to impregnation of tissues in live individuals. For example, the impregnation may be via infusion, or immersion techniques. The invention provides for the impregnation of materials that enter the tissues, organs and cells and allows for subsequent analysis that would not be possible without such impregnation.

The Perfusate

Fixatives

In one embodiment of the invention, the perfusate is a fixative, such as, but not limited to, an aldehyde (eg. paraformaldehyde, glutaraldehyde, formaldehyde, glyoxal); a ketone (eg. acetone, methyl ethyl ketone) or a low molecule weight alcohol (eg. ethanol, methanol, isopropanol, propanol, butanol, isobutanol, ethyl butanol, amyl alcohol). For example, if paraformaldehyde or glutaraldehyde is used as the fixative of choice, a 1-10% or greater solution is prepared in 50 mM sodium phosphate, pH 7.5 and the animal is perfused as described herein. (See also L. Angerer et al., Methods in Cell Biol. 35:37-71 (1991); U.S. Pat. No. 2,005,0153373 and U.S. Pat. No. 2,003,0064518) The skilled artisan would be cognizant of the preferred fixative for the particular studies being performed and the concentrations of fixative and the buffer necessary to achieve the desired effect.

Solvents

In another embodiment, the perfusate is a solvent. Depending on the procedure to be employed for subsequent analysis, the skilled artisan would be aware that the solvent used must be compatible with the analyte to be studied. Solvents of choice for MALDI, for example, would include dimethyl sulfoxide, (DMSO), methanol, ethanol, propylene glycol, ethylene glycol, polyethylene glycol, glycerol, beta-mercaptoethanol, or dimethyl formamide (DMF) (See U.S. Pat. Nos. 5,716,825; 5,705,813; 5,854,486; 5,808,300; 6,639,217; 6,677,161; 6,680,477; 6,706,530 and 6,723,564).

Buffers

In another embodiment, the perfusate is a buffered solution. As noted above, the buffer chosen for a particular analytical procedure following the perfusion would be prepared in accordance with the optimal pH of the analyte to be obtained from the tissue, organ or cell system under analysis. One of skill in the art would be cognizant of this fact.

Matrix Liquid

In another embodiment, the perfusate is a matrix liquid for use in matrix assisted laser desorption ionizing imaging (MALDI) mass spectrometry (MS). Examples of such matrices include α-4-cyano hydroxyl cinnamic acid (CHCA), sinnapinic acid, a heavy metal such as, but not limited to, gadolinium, cobalt and bismuth and glycerol. The choice of matrix depends on the system to be studied. For example, 2,5-dihydroxybenzoic acid (DHB) is often used with peptides, proteins, lipids and oligosaccharides. 3,5-dimethoxy-4-hydroxycinnamic acid is often used with peptides, proteins and glycoproteins. α-cyano-4-hydroxycinnamic acid (CHCA) is often used with peptides, proteins, lipids and oligonucleotides. The matrix may be prepared at a concentration of about 10 mM, although this concentration may be modified depending on the circumstances presented. (See U.S. Pat. Nos. 5,716,825; 5,705,813; 5,854,486; 5,808,300; 6,639,217; 6,677,161; 6,680,477; 6,706,530 and 6,723,564).

Cryopreservatives

In another embodiment, the perfusate may be a cryopreservative, known to those skilled in the art, including, but not limited to, propylene glycol, ethylene glycol, trialose, sucrose, glycerol, and a bisaccharide. The skilled artisan would be cognizant of the preferred cryopreservative for the particular studies being performed and the concentrations of cryopreservative necessary to achieve the desired effect.

Imaging/Contrast Media

In yet another embodiment, the perfusate is an imaging or contrast medium, such as those utilized in radiographic imaging or ultrasound techniques. Examples of these may be found in U.S. 20050180920; 20050036946 and 20050025711.

In addition, there are commercially available oral contrast agents currently available in liquid form containing non-toxic salts of diatrizoic acid, meglumine diatrizoate and sodium diatrizoate in an aqueous solution. Commercial preparations include Gastrografin sold by Bracco Diagnostics, Inc. of Milan, Italy, and Gastroview sold by Mallinckrodt, Inc. of St. Louis, Mo. Both products are dispensed in aqueous solution containing approximately 660 milligrams of meglumine diatrizoate and 100 milligrams of sodium diatrizoate per milliliter of solution. The recommended dosage of these salts for computerized tomographic examinations is 25 milliliters of contrast (containing 9.17 grams of iodine) in 1000 milliliters of water, which is administered orally approximately 15 to 30 minutes prior to imaging of the gastrointestinal tract.

Pharmacologically acceptable and non-toxic salts of diatrizoic acid are referenced in the US Pharmacopeia and comprise meglumine diatrizoate and sodium diatrizoate. Meglumine diatrizoate is designated chemically as 1-deoxy-1-(methylamino)-D-glucitol 3,5-diacetamido-2,4,6-triodobenzoate. Sodium diatrizoate is designated chemically as monosodium 3,5-diacetamido-2,4,6-triiodobenzoate. The clinical pharmacology of diatrizoate salts for use as gastrointestinal contrast media is the high atomic weight of iodine, which produces adequate radiodensity for radiographic contrast of body tissues, and its poor absorption from the gastrointestinal tract. Sodium diatrizoate contains more iodine on a weight basis, and is therefore more effective as a radiographic contrast agent, but is limited in high doses by its toxicity. Meglumine diatrizoate contains less iodine, but its solutions tend to be more viscous and less toxic. Accordingly, combinations of meglumine diatrizoate and sodium diatrizoate are often used in combination. The oral compositions are administered orally, approximately 50 minutes prior to computerized axial tomographic examination of the appendix.

The radio opaque compounds reported in the art generally fall into two categories: ionic and non-ionic. The ionic monomeric compounds used as contrast media for intravascular use have an osmolarity seven to eight times that of normal human blood. Non-ionic compounds such as lohexol, lopamidol, metrizamide are formulated as less hyperosmolar solutions.

U.S. Pat. No. 5,746,998 titled “Targeted co-polymers for radiographic imaging” describes polymeric compounds such as diblock copolymers capable of forming micelles for medical imaging. In addition, water soluble biologically inert polymers such as polyethylene oxide or poly (vinyl pyrrolidinone), with molecular weight above 20,000 g/mol are also used for radiographic imaging, although they are not eliminated by the body. Thus high molecular weight polymers above 20,000 are considered as non-degradable permanent implants. On the other hand, polyethylene glycol with a molecular weight below 300 is insoluble in water. Water solubility is considered essential for safe removal of the compound. Many derivatives of polyethylene oxide such as polyethylene glycol succinate based derivatives, glutaric acid based derivatives and hydroxy acid based derivatives are also used, but these undergo substantial hydrolysis and degradation when stored in water for prolonged periods of time.

Among the biodegradable polymers used for radioimaging, polymers prepared from hydroxy acids and/or polylactones have received much attention due to their degradability and toxicological safety. Homopolymers and copolymers based on the I-lactic acid, di-lactic acid and glycolic acid are among the most widely used polymers for medical applications. These polymers can be formulated into variety of physical forms such as fibers or filaments with acceptable mechanical properties, degradation profile and non-toxic degradation products.

To visualize the deployment of bioabsorbable implantable devices in the human or animal body, many surgical procedures are performed with the aid of fluoroscopic angiography. However, most biodegradable polymers used in current clinical practice have poor visibility when viewed using standard medical imaging equipment. The absorbable polymeric material may be visualized if they are radio-opaque and offer radiographic contrast relative to the body. To make the absorbable polymer radio-opaque, it must be made from a material possessing radiographic density higher than surrounding host tissue, and have sufficient thickness to affect the transmission of radiations and produce a contrast in the image. To improve the visualization, the biodegradable polymer must be chemically and physically modified. U.S. Pat. No. 6,174,330 titled “Bioabsorbable marker having radio-opaque constituents” discloses use of bioabsorbable polymer mixed with non-absorbable radio-opaque moieties such as heavy metal compounds mixed with the absorbable polymer. U.S. Pat. No. 6,475,477 titled “Radio-opaque polymer biomaterials” discloses tyrosine derived radio-opaque polymers.

Other contrast agents suitable for use in embodiments of the present invention may include paramagnetic lanthanide chelates and/or paramagnetic lanthanide linked to a macromolecule, such as gadolinium DPTA. Other examples of MR contrast for perfusion imaging include the application of susceptibility agents containing iron oxide or dysprosium that introduce local inhomogeneity into the magnetic field by causing large fluctuations in the magnetic moment between blood and intracellular compartments.

Biomarkers

In another embodiment, compounds including, but not limited to, small molecular weight (i.e. <2 kDa) peptides or other chemical compounds or markers may also be perfused to localize and quantitate the compounds of interest in or on the tissue samples. Accordingly, the perfusate may contain a biomarker for use in a diagnostic or therapeutic setting. The biomarker may be a protein, such as an antibody molecule, it may be a nucleotide, including DNA or RNA, or an antisense molecule or a siRNA, or a carbohydrate, a lipid, or any combination thereof. The biomarker may be labeled with a fluorophore, a radionuclide, a heavy metal, or an enzyme.

For example, U.S. patent publication 20050214300 describes the use of antibodies to Porimin, a protein expressed on many cancer cells in vivo (including breast, prostate, thyroid, kidney, lung, and ovarian cancer cells) to diagnose patients having this disease and to inhibit the proliferation of cells expressing this protein. Another target for diagnosing and treating prostate cancer is described in U.S. Pat. No. 6,835,822, whereby the invention discloses the use of the polynucleotide and polypeptide sequences of SGP28 for diagnosing patients having cancer cells expressing this molecule, particularly prostate cancer.

U.S. Pat. No. 6,929,797 discloses the use of conjugates of vitamin D compounds or analogs and a targeting molecule that allows for site-specific delivery of the vitamin D. Particular conjugates include a bone-therapeutic conjugate and an anti-tumor conjugate. Other therapeutic drugs may be radiolabeled and injected using the methods of the present invention.

Furthermore, U.S. Pat. No. 6,872,381 is directed to radiopharmaceuticals that bind the CCR1 receptor and are able to pass through the blood-brain barrier and are therefore useful in diagnosing Alzheimer's disease. In addition, U.S. Pat. No. 6,821,504 discloses an in vivo method for diagnosing Alzheimer's disease using magnetic resonance micro-imaging. Any targeting strategy may be used with the methods of the present invention. The methods of the present invention would thus allow for more accurate assessment of whether the agents to be delivered or targeted (as described above) achieved their target site.

Antibodies Used for Diagnostic Purposes

Various procedures known in the art may be used for the production of polyclonal antibodies to a target protein or other antigenic molecules. For the production of an antibody, various host animals can be immunized by injection with the antigen, including but not limited to rabbits, mice, rats, sheep, goats, etc. In one embodiment, the antigen or a fragment thereof can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward an antigen, or analog, or derivative thereof, any technique that provides for the production of antibody molecules by continuous cell lines in culture may be used. These include but are not limited to the hybridoma technique originally developed by Kohler and Milstein [Nature, 256:495-497 (1975)], as well as the trioma technique, the human B-cell hybridoma technique [Kozbor et al., Immunology Today, 4:72 (1983); Cote et al., Proc. Natl. Acad. Sci. U.S.A., 80:2026-2030 (1983)], and the EBV-hybridoma technique to produce human monoclonal antibodies [Cole et al., in Monoclonal Antibodies and Cancer Therapy, Alan R. Liss, Inc., pp. 77-96 (1985)]. In an additional embodiment of the invention, monoclonal antibodies can be produced in germ-free animals utilizing recent technology [PCT/US90/02545]. In fact, according to the invention, techniques developed for the production of “chimeric antibodies” [Morrison et al., J. Bacteriol., 159:870 (1984); Neuberger et al., Nature, 312:604-608 (1984); Takeda et al., Nature, 314:452-454 (1985)] by splicing the genes from a mouse antibody molecule specific for GLI together with genes from a human antibody molecule of appropriate biological activity can be used; such antibodies are within the scope of this invention. Such human or humanized chimeric antibodies are preferred for use in therapy of human diseases or disorders (described infra), since the human or humanized antibodies are much less likely than xenogenic antibodies to induce an immune response, in particular an allergic response, themselves.

According to the invention, techniques described for the production of single chain antibodies [U.S. Pat. Nos. 5,476,786 and 5,132,405 to Huston; U.S. Pat. No. 4,946,778] can be adapted to produce e.g., GLI-specific single chain antibodies. An additional embodiment of the invention utilizes the techniques described for the construction of Fab expression libraries [Huse et al., Science, 246:1275-1281 (1989)] to allow rapid and easy identification of monoclonal Fab fragments with the desired specificity for an antigen, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of the antigen, one may assay generated hybridomas for a product which binds to the antigen or a fragment containing such epitope and choose those which do not cross-react with the antigen. For selection of an antibody specific to the antigen from a particular source, one can select on the basis of positive binding with the antigen expressed by or isolated from that specific source.

The foregoing antibodies can be used in methods known in the art relating to the localization and activity of the antigen, e.g., for Western blotting, imaging the antigen in situ, measuring levels thereof in appropriate physiological samples, etc. using any of the detection techniques mentioned herein or known in the art. The standard techniques known in the art for immunoassays are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984) J. Clin. Chem. Clin. Biochem. 22:895-904.

One aspect of the invention provides using the methods of the invention for aid in the diagnosis or therapy of cancers or hyperproliferative diseases. The use of an antibody to a particular epitope on the cancer cell provides a general biomarker for the tumors, and may allow for tracking the presence of cancer cells, or allow for more accurate prognosis or to determine whether a particular therapy was effective in eradicating a cancer. Thus, the antibody compositions and methods provided herein, when delivered using the methods of the invention are particularly deemed useful for the diagnosis of tumors including those that may have metastasized.

The diagnostic method of the invention provides injecting the perfusate, which in this case is the labeled anti-tumor antibody and tracking the location of the labeled antibody by any imaging method described herein. The antibody is allowed to bind to the antigen to form an antibody-antigen complex. The conditions and time required to form the antibody-antigen complex in situ may vary and are dependent on the biological sample being tested and the method of detection being used.

The antibodies may be labeled with radioactive compounds, enzymes, biotin, or fluorochromes. Of these, radioactive labeling may be used for almost all types of antibodies. Enzyme-conjugated labels are particularly useful when radioactivity must be avoided or when quick results are needed. Biotin-coupled reagents usually are detected with labeled streptavidin. Streptavidin binds tightly and quickly to biotin and may be labeled with radioisotopes or enzymes. Fluorochromes, although requiring expensive equipment for their use, provide a very sensitive method of detection. Those of ordinary skill in the art will know of other suitable labels which may be employed in accordance with the present invention. The binding of these labels to antibodies or fragments thereof may be accomplished using standard techniques such as those described by Kennedy, et al. [(1976) Clin. Chim. Acta 70:1-31], and Schurs, et al. [(1977) Clin. Chim Acta 81: 1-40]. Once the labeled antibodies are injected, the tissue are removed as described herein and assayed for the presence of the label.

In accordance with the diagnostic method of the invention, the presence or absence of the antibody-antigen complex is correlated with the presence or absence in the biological sample of the antigen, or a peptide fragment thereof. A biological sample containing elevated levels of said antigen is indicative of a cancer in a subject from which the biological sample was obtained. Accordingly, the diagnostic method of the invention may be used as part of a routine screen in subjects suspected of having a cancer or for subjects who may be predisposed to having a cancer. Moreover, the diagnostic method of the invention may be used alone or in combination with other well-known diagnostic methods to confirm the presence of a cancer.

The diagnostic method of the invention further provides that an antibody of the invention may be used to monitor the levels of the tumor antigen in patient samples at various intervals of drug treatment to identify whether and to which degree the drug treatment is effective in reducing or inhibiting hyperproliferation of cells. Furthermore, antigen levels may be monitored using an antibody of the invention in studies evaluating efficacy of drug candidates in model systems and in clinical trials. The antigens provide for surrogate biomarkers in biological fluids to non-invasively assess the global status of tumor cell proliferation. For example, using an antibody of this invention, antigen levels may be monitored in biological samples of individuals treated with known or unknown therapeutic agents or toxins. Persistently increased total levels of the tumor antigen in biological samples during or immediately after treatment with a drug candidate indicates that the drug candidate has little or no effect on cell proliferation. Likewise, the reduction in total levels of the tumor antigen indicates that the drug candidate is effective in reducing or inhibiting tumor cell proliferation. This may provide valuable information at all stages of pre-clinical drug development, clinical drug trials as well as subsequent monitoring of patients undergoing drug treatment.

Antibody Labels

The proteins of the present invention, antibodies to these proteins, and nucleic acids that hybridize to these proteins (e.g. probes) etc. can all be labeled. Suitable labels include enzymes, fluorophores (e.g., fluorescein isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), and chemiluminescent agents. When a control marker is employed, the same or different labels may be used for the receptor and control marker.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. Such labels may also be appropriate for the nucleic acid probes used in binding studies with the protein. In the instance where the label is an enzyme, detection may be accomplished by any of the presently utilized calorimetric, spectrophotometric, fluorospectrophotometric, amperometric or gasometric techniques known in the art. Further techniques for use with the methods of the present invention are described below.

Direct labels are one example of labels which can be used according to the present invention. A direct label has been defined as an entity, which in its natural state, is readily visible, either to the naked eye, or with the aid of an optical filter and/or applied stimulation, e.g. ultraviolet light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70:419-439 (1980) and in U.S. Pat. No. 4,857,453.

Suitable enzymes include, but are not limited to, alkaline phosphatase and horseradish peroxidase. In addition, the protein (such as an antibody) or a fragment thereof can be modified to contain a marker protein such as green fluorescent protein as described in U.S. Pat. No. 5,625,048 filed Apr. 29, 1997, WO 97/26333, published Jul. 24, 1997 and WO 99/64592 all of which are hereby incorporated by reference in their entireties.

Other labels for use in the invention include magnetic beads or magnetic resonance imaging labels for functional MRI.

As exemplified herein, proteins, including antibodies, can be labeled by metabolic labeling. Metabolic labeling occurs during in vitro incubation of the cells that express the protein in the presence of culture medium supplemented with a metabolic label, such as [³⁵S]-methionine or [³²P]-orthophosphate. In addition to metabolic (or biosynthetic) labeling with [³⁵S]-methionine, the invention further contemplates labeling with [¹⁴C]-amino acids and [³H]-amino acids (with the tritium substituted at non-labile positions), or any other label useful with magnetic resonance imaging methods.

Delivery of the Perfusate

Any of the materials described above may be perfused into an animal using the methods of the present invention. In one embodiment, the animal is a human. In another embodiment the animal is a non-human mammal, including rodents, such as mice, rats, gerbils, hamsters and the like. In another embodiment, the non-human mammal is a non-human primate such as a monkey. In yet another embodiment, the non-human mammal is a cat, a dog, a rabbit, a goat, a sheep, a horse, a pig, and a cow.

The perfusion volume in milliliters is determined by the size of the animal in grams. For example, for a thirty gram mouse, 1.0 milliliter of perfusate produces the desired effect. The skilled artisan would be cognizant of how to adjust accordingly for a larger or smaller animal.

In one embodiment of the invention, the method for perfusion of live tissues, organs and cells provides for inserting a needle into the left ventricle of the heart through a percutaneous puncture of the left lateral chest wall at the juncture of the anterior at about ¼ to ½ of the thorax and the posterior at about ½ to ¼ of the thorax in the anterior/posterior plane and at the juncture of the superior ⅔ to ¾ and the inferior ⅓ to ½ of the distance between the axilla and the inferior margin of the rib cage in the cranio-caudal plane. The perfusate is delivered into an animal for a time period ranging from about 10 seconds to less than or equal to one minute. The bodily tissue is then removed for analysis.

In a more preferred embodiment, the point of needle insertion is at the juncture of the anterior one third and posterior two thirds of the thorax in the anterior/posterior plane, and at the juncture of the superior two thirds and the inferior one third of the distance between the axilla and the inferior margin of the rib cage in the cranio-caudal plane. The tissue can be harvested in about 90 to 120 seconds. The preferred embodiments do not require that a thoracotomy be performed.

The impregnation of cells and tissues in living subjects or the impregnation of removed tissues allows the penetration of the material to be examined to be done throughout the specimen rather than the present method which requires the post-fixation covering of parts of the tissue and analysis only of the surface that is covered, in distinction to the impregnation method which allows sampling and direct analysis of the complete sample. The advantages are illustrated by the effect of the use of the novel perfusion method, described herein.

For example, the perfusion impregnation method described in the present invention is minimally disruptive to the subject. The method allows the heart to perfuse the liquid at physiologic blood pressure and heart rate. This impregnation method achieves uniform distribution and impregnation of perfusate throughout all the tissues, organs, and cells of the body. This impregnation perfusion method allows physiologic uniform fixation of body tissues, organs, and cells without distortion using, but not limited to, such fixative perfusates as paraformaldehyde, glutaraldehyde, and alcohol. This perfusion method also allows uniform perfusion and impregnation of body tissues, organs, and cells with matrix and solvents including, but not limited to alpha 4-cyano hydroxy cinnamic acid, sinnapinic acid glycerol, and DMSO, for matrix assisted laser desorption ionization (MALDI) imaging of tissue, organ, and cell proteins, peptides, and other molecules. This perfusion method also allows uniform distribution of drugs in body tissues, organs, and cells. In addition, to the inventors' knowledge, this perfusion method is a novel method of physiologic perfusion of MALDI matrix liquids in living tissues prior to fixation.

Thus, the applications for this invention include, but are not limited to uniform physiologic perfusion fixation of living tissue, organs, and cells for examination by various types of microscopy, including, but not limited to light, electron, and atomic microscopy. The procedures described herein also provide for uniform physiologic perfusion of other drugs and chemicals throughout the tissues, organs, and cells of the body, before or after they are harvested for study. The methods described herein provide for tissue, organ, and cell impregnation with specialized materials including, but not limited to matrix, fixative, drugs, and other chemical compounds for analysis by specialized methods including, but not limited to Maldi imaging.

The preservation of the tissue or cellular architecture after using the perfusion methods of the invention may be monitored by standard immunohistochemistry procedures known to those skilled in the art, eg. through the use of stains such as Hematoxylin and Eosin and antibodies specific for cellular proteins, eg. Glial Fibrillary Acidic Protein (GFAP). Other staining procedures or antibodies useful for monitoring tissue integrity may be used based on the specific tissue being analyzed. For example, when analyzing particular immune cells in the spleen, one may use labeled antibodies specific for T cells, such as anti-CD4 and anti-CD8 antibodies, or for B cells, one may use labeled anti-Ig antibodies. If one were studying the levels of beta amyloid or of Tau protein in the brain tissue of Alzheimer's subjects, one may use anti-Tau or anti-beta amyloid antibodies.

Imaging Methods

The perfusion methods of the present invention provide for the preparation of tissues, organs or cells to be subsequently analyzed by any imaging or diagnostic protocol of one's choosing.

For example, embodiments of the present invention may also be used with molecular imaging strategies, for example, directing the contrast with molecular recognition sites to areas of tissue and quantifying the presence of a target or molecular process. Thus, particular embodiments of the present invention may have application in detecting cancer, inflammation, infection, swelling or edema, scar tissue, etc. Also, embodiments of the present invention could be used to define metabolic pathways that are functioning within tissue in an organ system. Particular embodiments of the present invention provide for the detection of tissue injury utilizing non-invasive imaging after administration of a contrast agent.

Non-invasive techniques suitable for use in embodiments of the present invention include Magnetic Resonance Imaging (MRI), ultrasound, x-ray computed tomography (CT), single photon emission computed tomography (SPECT) and/or positron emission tomography (PET). Comparisons may be made between a first or baseline image and a second image and the contrast of the image analyzed to detect the presence of tissue injury. As used herein, the term image refers to a spatial signal that may be evaluated to obtain a desired measure of signal intensity.

Subsequently, MALDI-TOF mass spectrometry (Biflex and Autoflex MALDI-TOF mass spectrometers (Bruker Daltonics) can be used) and SpectroTYPER RT™ software (Sequenom, Inc.) can be used to analyze and interpret the SNP genotype for each sample.

A laser desorption time-of-flight mass spectrometer may be used in embodiments of the invention. In laser desorption mass spectrometry, a substrate or a probe comprising markers is introduced into an inlet system. The markers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry, or MALDI-MS, is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry. MALDI-MS is useful for detecting the biomarkers of the invention if the complexity of a sample has been substantially reduced using the preparation methods described above.

Surface-enhanced laser desorption/ionization mass spectrometry, or SELDI-MS represents an improvement over MALDI for the fractionation and detection of biomolecules, such as proteins, in complex mixtures. SELDI is a method of mass spectrometry in which biomolecules, such as proteins, are captured on the surface of a protein biochip using capture reagents that are bound there. Typically, non-bound molecules are washed from the probe surface before interrogation. SELDI technology is available from Ciphergen Biosystems, Inc., Fremont Calif. as part of the ProteinChip®. System. ProteinChip® arrays are particularly adapted for use in SELDI. SELDI is described, for example, in: U.S. Pat. No. 5,719,060 (“Method and Apparatus for Desorption and Ionization of Analytes,” Hutchens and Yip, Feb. 17, 1998) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Xenogen provides the VivoVision imaging method, which utilizes in vivo biophotonic imaging, which non-invasively illuminates and monitors biological processes taking place in a living mammal in real time. In this procedure, luciferase is incorporated into cells and animals. Once it is activated, light is emitted and VivoVision captures this image and analyzes it. This procedure may be used to track gene expression, or the spread of disease or the effect of a new drug candidate.

Markers on the substrate surface can be desorbed and ionized using gas phase ion spectrometry. Any suitable gas phase ion spectrometers can be used as long as it allows markers on the substrate to be resolved. Preferably, gas phase ion spectrometers allow quantitation of markers.

In one embodiment, a gas phase ion spectrometer is a mass spectrometer. In a typical mass spectrometer, a substrate or a probe comprising markers on its surface is introduced into an inlet system of the mass spectrometer. The markers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of markers or other substances will typically involve detection of signal intensity. This, in turn, can reflect the quantity and character of markers bound to the substrate. Any of the components of a mass spectrometer (e.g., a desorption source, a mass analyzer, a detector, etc.) can be combined with other suitable components described herein or others known in the art in embodiments of the invention.

In another embodiment, an ion mobility spectrometer can be used to detect markers. The principle of ion mobility spectrometry is based on different mobility of ions. Specifically, ions of a sample produced by ionization move at different rates, due to their difference in, e.g., mass, charge, or shape, through a tube under the influence of an electric field. The ions (typically in the form of a current) are registered at the detector which can then be used to identify a marker or other substances in a sample. One advantage of ion mobility spectrometry is that it can operate at atmospheric pressure.

In yet another embodiment, a total ion current measuring device can be used to detect and characterize markers. This device can be used when the substrate has only a single type of marker. When a single type of marker is on the substrate, the total current generated from the ionized marker reflects the quantity and other characteristics of the marker. The total ion current produced by the marker can then be compared to a control (e.g., a total ion current of a known compound). The quantity or other characteristics of the marker can then be determined.

In another embodiment, an immunoassay can be used to detect and analyze markers in a sample. This method comprises: (a) providing an antibody that specifically binds to a marker; (b) contacting a sample with the antibody; and (c) detecting the presence of a complex of the antibody bound to the marker in the sample. The preparation of antibodies to an antigen and the methods for labeling such antibodies has been described above. After the antibody is provided, a marker can be detected and/or quantified using any of suitable immunological binding assays known in the art (see, e.g., U.S. Pat. Nos. 4,366,241; 4,376,110; 4,517,288; and 4,837,168). Useful assays include, for example, an enzyme immune assay (EIA) such as enzyme-linked immunosorbent assay (ELISA), a radioimmune assay (RIA), a Western blot assay, or a slot blot assay. These methods are also described in, e.g., Methods in Cell Biology: Antibodies in Cell Biology, volume 37 (Asai, ed. 1993); Basic and Clinical Immunology (Stites & Terr, eds., 7th ed. 1991); and Harlow & Lane, supra.

The methods for detecting these markers in a sample have many applications. For example, one or more markers can be measured to aid human cancer diagnosis or prognosis. In another example, the methods for detection of the markers can be used to monitor responses in a subject to cancer treatment. In another example, the methods for detecting markers can be used to assay for and to identify compounds that modulate expression of these markers in vivo or in vitro.

Data generated by desorption and detection of markers can be analyzed using any suitable means. In one embodiment, data is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of markers detected, including the strength of the signal generated by each marker.

Data analysis can include the steps of determining signal strength (e.g., height of peaks) of a marker detected and removing “outliers” (data deviating from a predetermined statistical distribution). The observed peaks can be normalized, a process whereby the height of each peak relative to some reference is calculated. For example, a reference can be background noise generated by instrument and chemicals (e.g., energy absorbing molecule) which is set as zero in the scale. Then the signal strength detected for each marker or other biomolecules can be displayed in the form of relative intensities in the scale desired (e.g., 100). Alternatively, a standard (e.g., a serum protein) may be admitted with the sample so that a peak from the standard can be used as a reference to calculate relative intensities of the signals observed for each marker or other markers detected.

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique markers and markers which are up- or down-regulated between samples. Marker profiles (spectra) from any two samples may be compared visually. In yet another format, Spotfire Scatter Plot can be used, wherein markers that are detected are plotted as a dot in a plot, wherein one axis of the plot represents the apparent molecular of the markers detected and another axis represents the signal intensity of markers detected. For each sample, markers that are detected and the amount of markers present in the sample can be saved in a computer readable medium. This data can then be compared to a control (e.g., a profile or quantity of markers detected in control, e.g., women in whom human cancer is undetectable).

Any suitable samples can be obtained from a subject to detect markers. Preferably, a sample is a blood serum sample from the subject. If desired, the sample can be prepared to enhance detectability of the markers. For example, to increase the detectability of markers, a blood serum sample from the subject can be preferably fractionated by, e.g., Cibacron blue agarose chromatography and single stranded DNA affinity chromatography, anion exchange. chromatography and the like. Sample preparations, such as pre-fractionation protocols, is optional and may not be necessary to enhance detectability of markers depending on the methods of detection used. For example, sample preparation may be unnecessary if antibodies that specifically bind markers are used to detect the presence of markers in a sample.

Any suitable method can be used to detect a marker or markers in a sample. For example, gas phase ion spectrometry or an immunoassay can be used as described above. Using these methods, one or more markers can be detected. Preferably, a sample is tested for the presence of a plurality of markers. Detecting the presence of a plurality of markers, rather than a single marker alone, would provide more information for the diagnostician. Specifically, the detection of a plurality of markers in a sample would increase the percentage of true positive and true negative diagnoses and would decrease the percentage of false positive or false negative diagnoses.

The detection of the marker or markers is then correlated with a probable diagnosis of human disease, such as cancer. In some embodiments, the detection of the mere presence or absence of a marker, without quantifying the amount of marker, is useful and can be correlated with a probable diagnosis of human disease. In other embodiments, the detection of markers can involve quantifying the markers to correlate the detection of markers with a probable diagnosis of human disease. Thus, if the amount of the markers detected in a subject being tested is higher compared to a control amount, then the subject being tested has a higher probability of having a human disease.

Similarly, in another embodiment, the detection of markers can further involve quantifying the markers to correlate the detection of markers with a probable diagnosis of human disease, such as cancer, wherein the markers are present in lower quantities in blood serum samples from human cancer patients than in blood serum samples of normal subjects. Thus, if the amount of the markers detected in a subject being tested is lower compared to a control amount, then the subject being tested has a higher probability of having a human cancer.

When the markers are quantified, it can be compared to a control. A control can be, e.g, the average or median amount of marker present in comparable samples of normal subjects in whom human cancer is undetectable. The control amount is measured under the same or substantially similar experimental conditions as in measuring the test amount. For example, if a test sample is obtained from a subject's blood serum sample and a marker is detected using a particular probe, then a control amount of the marker is preferably determined from a serum sample of a patient using the same probe. It is preferred that the control amount of marker is determined based upon a significant number of samples from normal subjects who do not have human cancer so that it reflects variations of the marker amounts in that population.

Data generated by mass spectrometry can then be analyzed by a computer software. The software can comprise code that converts signal from the mass spectrometer into computer readable form. The software also can include code that applies an algorithm to the analysis of the signal to determine whether the signal represents a “peak” in the signal corresponding to a marker of this invention, or other useful markers. The software also can include code that executes an algorithm that compares signal from a test sample to a typical signal characteristic of “normal” and human cancer and determines the closeness of fit between the two signals. The software also can include code indicating which the test sample is closest to, thereby providing a probable diagnosis.

### EXAMPLES

The following examples are put forth so as to provide those of ordinary skill in the art with a complete disclosure and description of how to use the methods of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Minimally-Invasive Method for Murine Brain Fixation

Materials and Methods

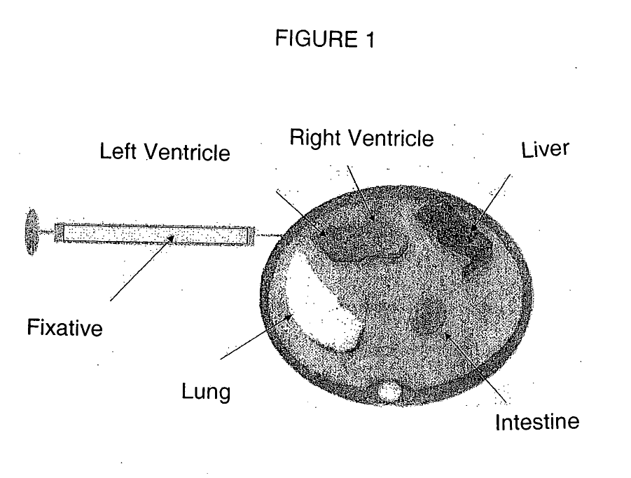

Perfusion Impregnation Method. A fine needle is inserted into the left ventricle of the heart through a percutaneous puncture of the left lateral chest wall. The point of needle insertion is at the juncture of the anterior one third and posterior two thirds of the thorax in the anterior/posterior plane, and at the juncture of the superior two thirds and the inferior one third of the distance between the axilla and the inferior margin of the rib cage in the cranio-caudal plane (FIG. 1).

All research was conducted under an approved protocol of the New York University Institutional Animal Care and Use Committee. All procedures were performed in accordance with protocols approved by New York University School of Medicine Institutional Animal Care and Use Committee. All procedures were carried out by trained personnel who continuously monitored the status of the animals. The mice were maintained in accordance with the National Institutes of Health Guide for the Care and Use of Laboratory Animals.

Fifteen male and female brown and white Swiss mice, average weight 30 g, were studied. The technique described below represents a procedure developed by the authors from prior experimental studies of rodent stroke (Eichenbaum, J. W., P. H. Pevsner, G. Pivawer, G. M. Kleinman, L. Chiriboga, A. Stern, A. Rosenbach, K. Iannuzzi and D. C. Miller. (2002); A murine photochemical stroke model with histologic correlates of apoptotic and nonapoptotic mechanisms. Journal of Pharmacological & Toxicological Methods 47:67-71; Pevsner, P. H., J. W. Eichenbaum, D. C. Miller, G. Pivawer, K. D. Eichenbaum, A. Stern, K. L. Zakian and J. A. Koutcher. (2001); A photothrombotic model of small early ischemic infarcts in the rat brain with histologic and MRI correlation. Journal of Pharmacological & Toxicological Methods 45:227-233).

The mice were immobilized with inhalation (Isoflurane 1.5%) anesthesia supplied by a face mask using the VetEquip, Inc. anesthesia machine (Pleasanton, Calif.). Anesthesia was administered continuously. The animals were maintained in a plane of surgical anesthesia (Martin, B. (1995). Institutional Care and Use Committee Anesthesia, Analgesia and Euthanasia Guide.)

A 27 gauge needle affixed to a tuberculin syringe loaded with tissue fixative was percutaneously inserted into the left ventricle. A solution of picric acid paraformaldehyde-glutaraldehyde was used so that both light and electron microscopy sections could be prepared from the same tissue specimen (Somogyi, P. and H. Takagi. (1982); A note on the use of picric acid-paraformaldehyde-glutaraldehyde fixative for correlated light and electron microscopic immunocytochemistry. Neuroscience 7:1779-1783). The needle orientation was perpendicular to the lateral chest wall and parallel to the sternum. The needle was inserted at the junction between the anterior one third and posterior two thirds of the lateral chest wall. The appearance of blood in the needle hub confirmed the correct position of the needle tip. A one milliliter volume of fixative was injected over 30 seconds. The fixative infusion resulted in post-brain perfusion cardiac arrest.

The brain was then removed intact as follows: A vertical midline scalp incision extending from the level of the cervical-cranial junction to the tip of the nose was made with a number 12 scalpel blade. The calvarium between the eyes was pierced with a straight, fine scissors. The scissors blades were quickly opened, splitting the calvarium in the midline. The skull edges were retracted and the brain removed intact. The entire brain harvest can be achieved in under 60 seconds. The fixed brain is ready for further study.

Results and Discussion

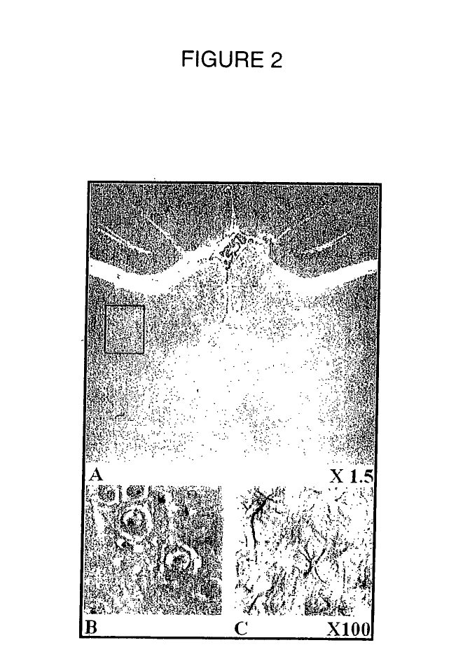

Proper tissue fixation (FIG. 2) was confirmed by light microscopy on coronal sections that were stained with Hematoxylin and Eosin (H&E), and by standard immunohistochemisty with an anti-Glial Fibrallary Acidic Protein (GFAP) antibody.

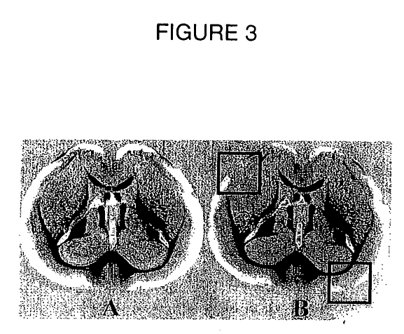

In the high power light microscopic examination the architecture and cytology of the grey and white matter components neuropil, neurons, and glia are well preserved (FIG. 2B). The ventricles were normal in size and not dilated (FIG. 3A).

Sample localization is paramount for identification of tissue pathologies using EM. The tissue punch technique on histologic specimens (FIG. 3) precisely identifies the region of sampling. In FIG. 3B, the punched sampling sites for electron microscopy obtained in this report are identified in black squares.

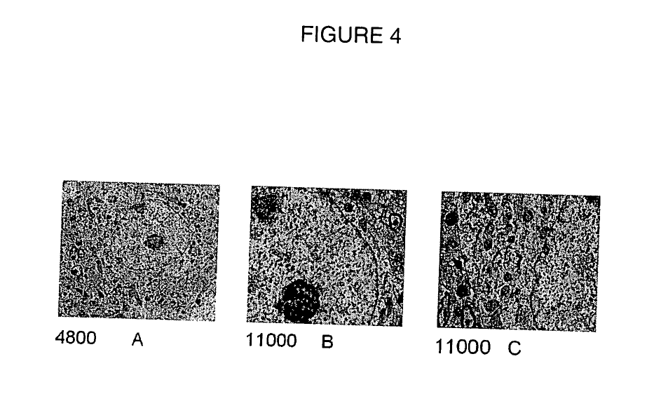

Ultrastructural regions of interest from the punched samples are displayed in FIG. 4. Ultrastructural preservation was confirmed by electron microscopy. Note that the cytoplasmic elements including mitochondria, golgi apparatus, and endoplasmic reticulum are intact. Both the cytoplasmic and nuclear membranes are undisturbed. The nuclear DNA maintains its normal appearance. In no instance were the mitochondria swollen or the mitochondrial cristae thickened, as would be expected in early necrosis (8).

Trans-thoracic, left ventricular cardiac injection of fixative during anesthesia produces complete tissue fixation without distortion.

The success of this method relies on the proper placement of the needle at the chest wall and insertion into the left ventricle. Other approaches to the left ventricle may involve inadvertent injection of fixative into the right ventricle. In mice the right ventricle is directly anterior to the left ventricle. Avoidance of the right ventricle is important to prevent tears in the thinner right ventricular wall, causing failure of the procedure and premature death of the animal. Thus, we stress the importance of needle placement and lateral left ventricular entry.

With the minimally-invasive method described herein, pre-fixation perfusion with saline is unnecessary and the animal's heart pumps the fixative into the brain, completely fixing all tissue. Injection of one milliliter of fixative into the mouse circulation over 30 seconds results in complete brain fixation without artifact. This method allows the physiologic blood pressure to perfuse the brain and avoids pressure artifact of brain swelling and ventricular enlargement. This technique allows for the examination of acute, discrete change in brain tissue.

Summary

Complete brain fixation can be achieved with transthoracic cardiac infusion without thoracotomy. Light and electron microscopy tissue sections reveal preservation of cytoplasmic and nuclear structure at all magnification levels. Punched samples were obtained from the fixed tissue specimens in precisely localized areas for study by electron microscopy. This perfusion fixation technique provides both faster tissue harvesting capability and higher quality tissue preservation, without the artifacts of brain swelling and ventricular dilation observed in direct cardiac perfusion. Acute, discrete change in brain tissue can be studied.

We claim:

1. A minimally invasive method for preparation of live body tissues for examination, comprising transthoracic cardiac infusion of a tissue perfusate in an amount sufficient to preserve the ultrastructure of the tissue without inducing artifacts.

2. The method of claim 1, wherein the transthoracic cardiac infusion comprises the steps of:
a. inserting a needle into the left ventricle of the heart through a percutaneous puncture of the left lateral chest wall at the juncture of the anterior at about ¼ to ½ of the thorax and the posterior at about ½ to ¾ of the thorax and in the anterior/posterior plane and at the juncture of the superior ⅔ to ¾ and the inferior ⅓ to ½ of the distance between the axilla and the inferior margin of the rib cage in the cranio-caudal plane;
b. delivering a perfusate using the method of step (a) into an animal for a time period ranging from about 10 seconds to less than or equal to one minute;
c. removing a bodily tissue for analysis.

3. The method of claim 2, wherein the transthoracic cardiac infusion comprises insertion of the needle at the juncture of the anterior one third and posterior two thirds of the thorax in the anterior/posterior plane, and at the juncture of the superior two thirds and the inferior one third of the distance between the axilla and the inferior margin of the rib cage in the cranio-caudal plane.

4. The method of claim 1, wherein the tissue perfusate is a fixative, a solvent, a matrix liquid for use in matrix assisted laser desorption ionization imaging (MALDI), a radionuclide, an imaging or contrast agent, a radiographic contrast medium, a cryopreservative, a biomarker, or a buffered solution for delivery of a therapeutic or diagnostic agent.

5. The method of claim 4, wherein the fixative is selected from the group consisting of paraformaldehyde, glutaraldehyde, formaldehyde, glyoxal, ethanol, methanol, propanol, isopropanol, butanol, isobutanol, ethyl butane, amyl alcohol, acetone and methyl ethyl ketone.

6. The method of claim 4, wherein the buffered solution is selected from the group consisting of phosphate buffered saline (PBS), a phosphate buffer, a potassium buffer, a choline buffer and a glycine buffer.

7. The method of claim 4, wherein the cryopreservative is selected from the group consisting of propylene glycol, ethylene glycol, trialose, sucrose, glycerol and a bisaccharide.

8. The method of claim 4, wherein the matrix liquid or solvent for use in matrix assisted laser desorption ionization imaging (MALDI) is infused prior to or concurrent with the infusion of the fixative.

9. The method of claim 4, wherein the matrix liquid is selected from the group consisting of α-4-cyano hydroxy cinnamic acid (CHCA), sinnapinic acid, a heavy metal and glycerol.

10. The method of any one of claims 1, 2, or 3, wherein the method provides for perfusion of the perfusate at physiologic blood pressure and heart rate.

11. The method of any one of claims 1, 2, or 3, wherein the method provides for uniform distribution and impregnation of perfusate throughout all tissues, organs and cells of the body, without distortion.

12. The method of any one of claims 1, 2, or 3, wherein the method provides for targeting of a diagnostic or therapeutic agent to a tissue, cell or organ.

13. The method of claim 7, wherein the MALDI is used for the imaging of a tissue, an organ, a cellular protein, a peptide, a sugar, a salt, an organic acid or any other low molecular weight molecule.

14. The method of claim 4, wherein the solvent is dimethyl sulfoxide (DMSO), dimethyl formamide (DMF), polyethylene glycol, beta-mercaptoethanol, methanol, ethanol, propylene glycol, or ethylene glycol.

15. The method of any one of claims 1, 2, or 3, further comprising infusing of a drug, a protein, an antibody, a nucleic acid, a carbohydrate or a lipid.

16. The method of claim 14, wherein the drug, the protein, the antibody, the nucleic acid, the carbohydrate or the lipid is labeled for monitoring tissue, organ or cellular disposition or damage.

17. The method of claim 15, wherein the drug, the protein, the antibody, the nucleic acid, the carbohydrate or the lipid is labeled with a marker selected from the group consisting of a radioisotope, a fluorophore, an enzyme or a heavy metal.

18. The method of any one of claims 1, 2, or 3, wherein the examination of tissues may be performed by a method selected from the group consisting of light microscopy, electron microscopy, atomic microscopy, Magnetic Resonance Imaging (MRI), ultrasound, x-ray computed tomography (CT), single photon emission computed tomography (SPECT) and/or positron emission tomography (PET), mass spectrometry, matrix assisted laser desorption ionizing imaging (MALDI-MS) spectrometry, surface-enhanced laser desorption/ionization mass spectrometry (SELDI), in vivo biophotonic imaging (VivoVision) and any other imaging method suitable for studying organ, tissue or cellular ultrastructure.

19. The method of claim 16, wherein the labeled drug, protein, antibody, nucleic acid, carbohydrate or lipid is uniformly distributed throughout the tissues, organs or cells of the body, before they are harvested for study.
